(19) United States
(12) Patent Application Publication
(10) Pub. No.: US 2016/0206868 A1
(43) Pub. Date: Jul. 21, 2016

(54) VALVED MALE LUER CONNECTOR

(71) Applicant: INDUSTRIE BORLA S.P.A., Moncalieri (Torino) (IT)

(72) Inventor: Gianni GUALA, Torino (IT)

(73) Assignee: INDUSTRIE BORLA S.P.A., Moncalieri (Torino) (IT)

(21) Appl. No.: 15/002,063

(22) Filed: Jan. 20, 2016

(30) Foreign Application Priority Data
Jan. 21, 2015 (IT) TO2015A000045

(51) Int. Cl. A61M 39/10; A61M 39/26

(52) U.S. Cl. CPC A61M 39/10; A61M 39/26; A61M 39/1011; A61M 2039/265

(57) ABSTRACT

A valved male luer connector includes an elastic hollow element enclosing a tubular member having an inlet portion and an outlet portion. A collar is axially displaceable to cause a stretching deformation of the elastic hollow element and open a flow passage between the inlet portion and the outlet portion of the tubular member. The inlet portion is rotatable with respect to the casing in both directions of rotation.

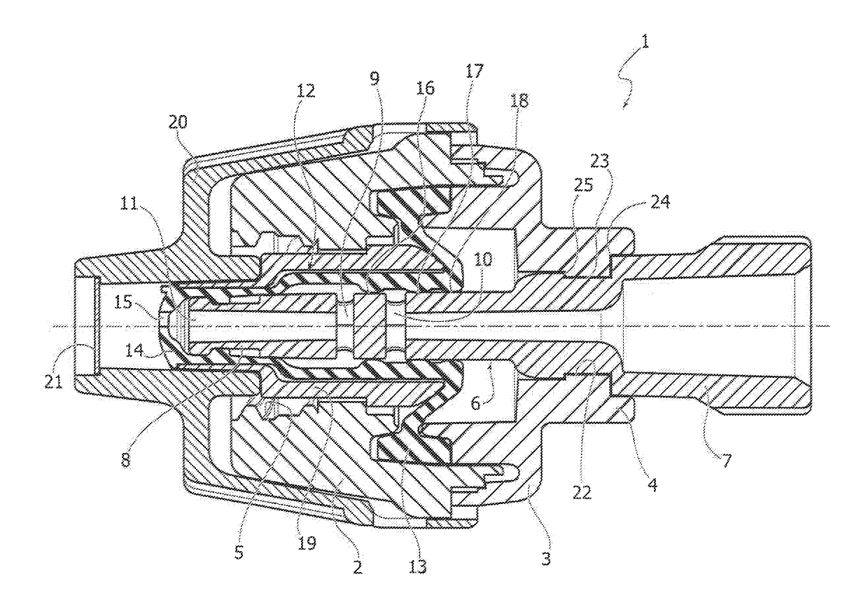

### CROSS REFERENCE TO RELATED APPLICATION

This application claims priority from Italian Patent Application No. TO2015A000045, filed on Jan. 21, 2015, the entire disclosure of which is incorporated herein by reference.

### FIELD OF THE INVENTION

The present invention generally refers to medical fluid connectors, and more in particular it regards a valved male luer connector applicable to a fluid line and adapted to be connected to a connector generally of the female type and the like for opening the flow passage through the two connectors.

### STATE OF THE PRIOR ART

A valved male luer connector comprising a casing, a tubular member having an inlet portion configured for connection to a medical line and an outlet portion with an open terminal end is known from European patent n° EP-2504056B1 in the name of the Applicant. An elastic hollow element anchored to the casing surrounds the tubular member and it has a terminal wall normally sealingly closing the terminal end of the outlet portion of the tubular member, and a cut. A collar is interposed between the casing and the elastic hollow element and it is axially displaceable, following the thrust engagement by a female connector that can be connected to the male connector, to cause a stretching deformation of the elastic hollow element with the consequent opening of the cut and thus the flow passage between the inlet portion and outlet portion of the tubular member.

### SUMMARY OF THE INVENTION

The present invention regards an improvement of the aforementioned known valved male luer connector, in particular as regards an easier and safer connection of the connector to the medical line.

According to the invention, this object is attained thanks to the fact that the inlet portion of the tubular member is rotatable with respect to the casing.

Thanks to this characteristic, when the valved male luer connector is connected, in use, to the medical line, typically constituted by a flexible duct, it can be manipulated so as to be connected to the female connector not only in an easier fashion, due to the fact that any twisting of the medical line is efficiently avoided, but also more safely since it avoids the risk of inadvertent detachment of the medical line due to a torsion thereof

The tubular member is freely rotatable with respect to the casing in both opposite directions of rotation, and according to a preferred embodiment of the invention, the tubular member of the connector is made in a single piece with the inlet portion thereof and it is rotatable as a whole with respect to the casing. In this case, the body of the connector has, on the side of such inlet portion, a hollow appendage through which the tubular member is rotatably engaged following an axial snap-fit coupling thereof during the assembly of the connector.

### BRIEF DESCRIPTION OF THE DRAWINGS

The invention will now be described in detail with reference to the FIGURE, provided purely by way of non-limiting example, representing an axial sectional view of the valved connector according to the invention.

### DETAILED DESCRIPTION OF THE INVENTION

With reference to the FIGURE, the valved male luer connector according to the invention comprises (as generally known from the aforementioned document EP-2504056B1) a casing 1 constituted by a hollow body 2 and an annular flange part 3 permanently axially coupled to the hollow body 2 and formed, on the opposite side, with a tubular appendage 4 to be described hereinafter.

The hollow body 2 has, on the side opposite to the flange 3, an internally threaded seat 5.

A tubular member which is axially extended through the body 1 is indicated in its entirety with 6 and it is rotatable with respect thereto through the methods described hereinafter.

The tubular member 6 has an inlet portion 7, preferably formed integrally joined and projecting to the external of the tubular appendage 4, configured for coupling a flexible duct of a medical line.

On the opposite side, the tubular member 6 has an outlet portion 8 which projects to the external of the threaded seat 5 to define a male luer lock connector adapted to be engaged by a supplementary female connector, not illustrated in that of the per se known type. For example, the female connector may be of the valved type described and illustrated in document EP-834665B1, also on behalf of the Applicant.

The inlet 7 and outlet 8 portions are connected to each other through a series of spaced radial passages 9, 10 whose mutual communication is controlled as clarified hereinafter, and the outlet portion 8 has an open terminal end indicated with 11.

Indicated with 12 is an elastic hollow element, typically made of elastomeric or silicone rubber, including an annular base 13 anchored in the casing 1 between the body 2 and the flange 3, and a terminal transversal wall 14 normally arranged facing the terminal end 11 of the tubular member 6 and having a cut 15 normally closed so as to substantially sealingly occlude the terminal end 11.

The elastic hollow element 12 internally has a first, a second and a third sealing annular relief respectively indicated with 16, 17 and 18 at contact with the tubular member 6. The annular relief 16 interacts with the passages 9 and 10 for respectively closing and opening the communication between the inlet portion 7 and the outlet portion 8 of the tubular member 6: in the condition represented in the FIGURE, the passages 9 and 10 are insulated with respect to each other.

A collar interposed between the body 2 of the casing 1 and the elastic hollow element 12 and constituting an actuator member adapted to control the opening of the flow passage through the connector due to a stretching deformation of the elastic hollow element 12 is indicated with 19. The collar 19 is predisposed to cooperate, as described in the previously mentioned document EP-2504056B1, with a female luer connector or the like coupled, in use, with the seat 5 of the body 2. Due to such coupling the female connector exerts an axial thrust against the collar 19 so as to push it in the direction of the inlet portion 7 and elastically stretch deforming the elastic hollow element 12, so as to open the flow passage through the cut 15.

A priming cap which can be removably applied to the casing 1 to keep the connector in a condition of at least partial opening before the coupling thereof with the female connector is indicated with 20. Thus, in use, the medical liquid coming from the duct connected to the tubular appendage 7 is capable of reaching the terminal end 11 of the tubular member 6 without requiring a priming intervention by the operator, thus making the valved male luer connector ready for coupling thereof to the female connector and allow the supply of the medical liquid to a patient. The priming cap 20, which is provided with a liquid-impermeable transversal barrier 21, is rotatably displaceable between a first axial position in which the valved connector remains closed, and a second axial position in which it interacts with the collar 19 so as to stretch deform the elastic element 12 freeing the communication between the radial passages 9 and 10 and thus between the inlet 7 and outlet 8 portions of the tubular member 6, simultaneously opening the cut 15. Thus, the inlet end 7 and the duct, in the use related thereto, are kept in communication with the atmosphere through the membrane 21 which, as mentioned, constitutes a barrier impermeable to the liquid coming from the duct. Thus, the connector according to the invention is ready for the subsequent coupling with the female connector without requiring further priming operations: as a matter of fact, it is sufficient to remove the cap 20 to be able to apply the female connector.

As mentioned previously, the tubular member 6 with the relative inlet portion 7 is, according to the distinctive characteristic of the invention, freely rotatable with respect to the casing 1 in both opposite directions of rotation. For this purpose, the tubular appendage 4 of the flange 3 is formed with an inner annular relief 22 with which there is engaged an annular groove 22 of the tubular member 6, formed between two axial stop steps 24, 25 of which the second, the innermost one, has a smaller diameter. Thus, during the assembly of the connector the tubular member 6 is axially inserted into the casing 1 up to obtaining a snap-fit coupling of the annular groove 23 with respect to the annular relief 22 of the flange 3. Following such snap-fit coupling, the tubular member 6 with the inlet portion 7 is rotatable and axially locked with respect to the casing 1. Thus, in use, the casing 1 is capable of rotating with respect to the duct of the medical line fixed to the inlet portion 7, thus being advantageous not only for an easier handling of the connector, but also as regards the safety thereof: The free rotation between the connector and the duct actually prevents the latter from twisting and possibly inadvertently separating from the connector.

Obviously, the construction details and the embodiments may widely vary with respect to what has been described and illustrated, without departing from the scope of protection of the present invention as defined in the claims that follow. Thus, for example, though in the described embodiment the inlet portion 7 or 7a is made of a single piece with the tubular member 6, it could also be separated and thus rotatable independently from such tubular member.

1. A valved male luer connector comprising:
a casing, a tubular member having an inlet portion configured for connection to a medical line and an outlet portion with a terminal open end,
an elastic hollow element secured to the housing and enclosing the tubular member, said elastic hollow element having a terminal wall normally sealingly closing said terminal end of the outlet portion of the tubular member and having a cut, and a collar interposed between the casing and the elastic hollow element,
said elastic hollow element axially displaceable, following thrust engagement by a female connector connectable to said male connector, to cause a stretching deformation of said elastic hollow element with the consequent opening of flow passage between said inlet portion and said outlet portion of the tubular member, wherein said inlet portion is rotatable with respect to the casing.

2. The connector according to claim 1, wherein said tubular member as a whole is rotatable with respect to the casing.

3. The connector according to claim 2, wherein said tubular member is freely rotatable with respect to the casing in opposite directions of rotation.

4. The connector according to claim 2, wherein the casing is provided, on the side of said outlet portion, with a hollow appendage through which said tubular member is rotatably engaged following an axial snap-fit coupling thereof.

5. The connector according to claim 1 further comprising a priming cap designed to be releasably connected to the casing to keep the outlet portion of the tubular member in communication with the atmosphere through a liquid-impermeable barrier.

6. The connector according to claim 2 further comprising a priming cap designed to be releasably connected to the casing to keep the outlet portion of the tubular member in communication with the atmosphere through a liquid-impermeable barrier.

7. The connector according to claim 3 further comprising a priming cap designed to be releasably connected to the casing to keep the outlet portion of the tubular member in communication with the atmosphere through a liquid-impermeable barrier.

8. The connector according to claim 4 further comprising a priming cap designed to be releasably connected to the casing to keep the outlet portion of the tubular member in communication with the atmosphere through a liquid-impermeable barrier.
